(19) United States
(12) Patent Application Publication
(10) Pub. No.: US 2018/0303519 A1
(43) Pub. Date: Oct. 25, 2018

(54) TRANSVERSE SHIFT SCREW TAIL, TRANSVERSE ADJUSTABLE SPINAL SCREW, AND IMPLANTATION METHOD

(71) Applicant: SHANGHAI SANYOU MEDICAL CO., LTD., SHANGHAI (CN)

(72) Inventors: MICHAEL MINGYAN LIU, SHANGHAI (CN); YONG QIU, SHANGHAI (CN); YUEMING SONG, SHANGHAI (CN); QIXIN CHEN, SHANGHAI (CN); JEAN-CHARLES LEHUEC, SHANGHAI (CN); RUIFENG LIU, SHANGHAI (CN)

(73) Assignee: SHANGHAI SANYOU MEDICAL CO., LTD., SHANGHAI (CN)

(21) Appl. No.: 15/765,121

(22) PCT Filed: Jun. 8, 2016
(86) PCT No.: PCT/CN2016/085194

§ 371 Date: Mar. 30, 2018

(30) Foreign Application Priority Data
Sep. 30, 2015 (CN) 201510642652X
Sep. 30, 2015 (CN) 2015207712882

(51) Int. Cl. A61B 17/70

(52) U.S. Cl. CPC A61B 17/7032; A61B 17/7041; A61B 17/7046

(57) ABSTRACT

A transverse shift screw tail, a transverse adjustable spinal screw, and an implantation method. The screw comprises a screw tail and a screw seat, the screw seat has a rod groove used for placing a connecting rod, which is recessed downwards from the top of the screw seat and radially penetrates the screw seat, a bottom of the rod groove is a curved surface adapted to an outer circumferential surface of the connecting rod, and the rod groove is provided with a locking thread fit with a locking bolt. The screw tail comprises a fixing column capable of being screwed into a vertebral body and a platform located at an end portion of the fixing column, the platform is provided with the installation head used for connecting with the screw seat, and an eccentric distance is provided between a center of the installation head and an axis of the fixing column.

### BACKGROUND OF THE PRESENT INVENTION

### Field of Invention

The present invention relates to a transverse shift screw tail.

The present invention further relates to a transverse adjustable spinal screw and an implantation method.

### Description of Related Arts

Spinal internal fixation technique was started to be applied since 1960s for treating various spinal diseases such as spine malformation, adolescent scoliosis, spinal fracture and spinal degeneration through operations. Spinal internal fixation technique may be briefly described as the following several steps: firstly, selecting a spinal segment which needs to be fixed, and implanting anchoring appliances (such as hooks or screws) into specific positions on all or partial vertebral bodies at two ends or in this segment; then locking connecting appliances (such as fixing steel plate or fixing rods) to each hook or screw to restrict relatively motion within the spinal segment such that the spinal segment is enabled to become a stable body; and finally, placing bone graft materials (such as autogenous bones or bone substitute materials) between vertebral bodies in this segment to expect that the spinal segment is gradually incorporated to form an integral body within a period of time after an operation, so as to guarantee long-term stability.

Each spine can be divided into an anterior portion and a posterior portion, the anterior portion is the portion towards the direction of abdomen, a posterior portion is the portion towards the direction of back, and the bone tissues connecting the anterior portion and the posterior portion are called as vertebral pedicles. All screws which enter bones from the posterior portion and are implanted into vertebral bodies through vertebral pedicles are collectively called as vertebral pedicle screws, and usually screw seat left at the posterior portion are provided with rod grooves for locking a connecting rod.

Vertebral pedicle screws are anchoring appliances which are the most commonly used at present in a spinal internal fixation system, and each vertebral pedicle screw consists of a screw tail (threaded portion implanted into a bone) and a screw seat (other portion left outside the bone). In general, vertebral pedicle screws are divided into fixed-angle screws (as illustrated in FIG. 1, wherein the screw tail and the screw seat are integrated) and variable-angle screws (as illustrated in FIG. 2, wherein the screw tail and the screw seat have a certain folding adjustable angle).

Usually a plurality of screws are used in one spinal fixation operation, the screws are respectively implanted into each vertebral body which needs to be fixed, and then a connecting rod is installed into each screw grooves and is locked to finally achieve the purpose of fixing a spine. Implantation of screws mainly considers anatomical characteristics of target vertebral bodies and restriction of an operation space, and thus positions and directions from which screws enter bones are possibly very different even though the screws are located between two adjacent vertebral bodies. Fixed-angle screws have the disadvantages that the angle on the lateral plan of spine cannot be adjusted, and vertebral bodies might be excessively bent or extended when the connecting rod is locked. While, by using adjustable-angle screws, this kind of problems can be avoided to a very great extent. However, on the front plan of a spine, screws implanted into different vertebral bodies are possibly dislocated in a left-right direction, and consequently it will cause a very great difficulty to installation of the connecting rod, especially when a plurality of screws are used. Usually, a doctor has to make some compromises such as linearly arranging screws, which will result in difficult to guarantee that each screw is implanted under an ideal state, or cooperatively connecting each screw seat by bending the connecting rod, which will make the operation difficulty and complex, and increase a certain uncertainty or cause improperly distorting of a spine.

Up to now, there have been no spinal internal fixation appliances and methods which can satisfactorily solve this problem.

### SUMMARY OF THE PRESENT INVENTION

In view of the above-mentioned disadvantages of the prior art, the purpose of the present invention is to provide a transverse shift screw tail, a transverse adjustable spinal screw, and an implantation method, which are used for solving the problem that it is difficult to linearly arrange a plurality of screws implanted into vertebral bodies in the prior art.

In order to realize the above-mentioned purposes and other related purposes, the present invention provides a transverse shift screw tail, the screw tail comprises a fixing column capable of being screwed into a vertebral body and a platform located at an end portion of the fixing column, the platform is provided with an installation head used for connecting with a screw sea, and an eccentric distance is provided between a center of the installation head and an axis of the fixing column.

Preferably, an eccentric distance between the center of the installation head and the axis of the fixing column ranges 1 mm-5 mm.

Preferably, an outer circumferential surface of the fixing column is provided with a thread.

Preferably, an outer surface of the installation head is a spherical surface and a top of the installation head is provided with an inwards recessed noncircular installation adjusting hole.

In order to realize the above-mentioned purposes and other related purposes, the present invention further provides a transverse adjustable spinal screw, the screw comprises the screw tail in the above-mentioned technical solution and a screw seat installed on an installation head of the screw tail, a top of the screw seat is provided with a rod groove used for placing a connecting rod, the rod groove is recessed downwards from the top of the screw seat and radially penetrates the screw seat, a bottom of the rod groove is a curved surface adapted to an outer circumferential surface of the connecting rod, and a side surface of the rod groove is provided with a locking thread fit with a locking bolt.

Preferably, the screw seat comprises a screw seat sleeve and a slidable tensioning ring which is sleeve-mounted into the screw seat sleeve, the slidable tensioning ring is sleeve-mounted onto the installation head of the screw tail, and the rod groove is located at one end, away from the slidable tensioning ring, of the screw seat sleeve.

More preferably, the slidable tensioning ring is provided with a ball socket for placing the installation head, the ball socket is provided with an opening facing to the installation head, and a diameter of the opening is smaller than a maximum diameter of the installation head.

More preferably, a circumferential surface of the ball socket is provided with a plurality of extension grooves which are uniformly distributed along a circumferential direction, and the extension grooves extend from the opening to an end corresponding to the opening.

More preferably, one end, facing to the screw tail, of an inner circumferential surface of the screw seat sleeve is provided with a limiting ring and an inner circumferential surface of the limiting ring is fit with an outer circumferential surface of the opening of the ball socket.

More preferably, a portion, corresponding to the ball socket, of the inner circumferential surface of the screw seat sleeve is provided with an inwards recessed loose section and a gap is kept between the loose section and an outer circumferential surface of the ball socket.

More preferably, a sidewall of the slidable tensioning ring is further provided with at least one limiting groove which extends downwards from a top, and the screw seat sleeve is provided with a limiting screw corresponding to the limiting groove.

More preferably, one end, facing to the rod groove, of the slidable tensioning ring is provided with an arc groove, an inner circumferential surface of the arc groove corresponds to the bottom of the rod groove, and when the slidable tensioning ring is installed in place, the inner circumferential surface of the arc groove protrudes out of the bottom of the rod groove.

In order to realize the above-mentioned purposes and other related purposes, the present invention further provides a method for implanting a transverse adjustable spinal screw into a vertebral body, the transverse adjustable spinal screw is the transverse adjustable spinal screw according to the above-mentioned technical solution, and the method comprises the following steps:

1) determining a screwing-in point P of each screw on the vertebral body and screwing the screw tail of each screw into the vertebral body from the preset screwing-in point P;

2) setting a placement position of the connecting rod on the vertebral body and marking the placement position of the connecting rod as a reference line L;

3) rotating the platform to enable the center of the installation head to approach the reference line L to the utmost extent;

4) installing the screw seat on the installation head of each screw tail and rotating the screw seats to enable the axes of the rod grooves of the screw seats to be located on the same line;

5) inserting the connecting rod into the rod grooves to enable all screw seats to be integrally connected; and

6) installing the locking bolt at the top of each screw seat, the locking bolt stretching into the rod groove and being fit with the locking thread in the rod groove.

As described above, the transverse shift screw tail, the transverse adjustable spinal screw, and the implantation method provided by the present invention have the following beneficial effects:

According to the transverse shift screw tail, the transverse adjustable spinal screw and the implantation method, by designing a brand new screw, the screw seats are connected linearly, the installation process of the connecting rod is enabled to be reliable and effective, a spine is prevented from being improperly distorted, the difficulty of operation by a doctor is reduced and the operation effect is improved.

### BRIEF DESCRIPTION OF THE DRAWINGS

FIG. 1 illustrates a structural schematic view of a fixed-angle screw in the prior art.

FIG. 2 illustrates a structural schematic view of a variable-angle screw in the prior art.

FIG. 3 illustrates a structural schematic view of a transverse shift screw tail in the present invention.

FIG. 4 illustrates a front view of the transverse shift screw tail illustrated in FIG. 3.

FIG. 5 illustrates a structural schematic view of a transverse adjustable spinal screw in the present invention.

FIG. 6 illustrates a structural schematic view of a screw seat of the transverse adjustable spinal screw illustrated in FIG. 5.

FIG. 7 illustrates a stereoscopic schematic view of the screw seat illustrated in FIG. 6.

FIG. 8 illustrates a structural schematic view of a slidable tensioning ring illustrated in FIG. 6.

FIGS. 9-14 illustrate schematic views of steps of a method for implanting the transverse adjustable spinal screw illustrated in FIG. 5.

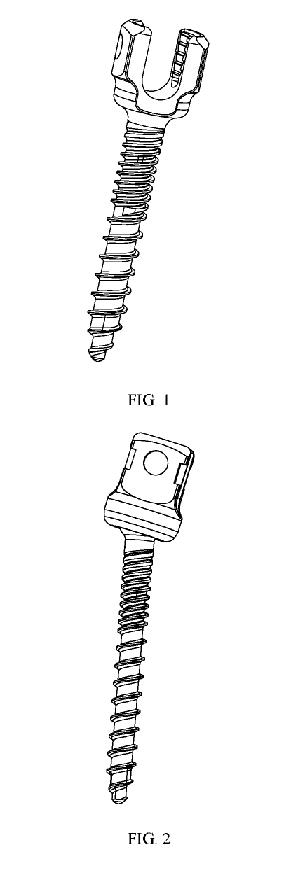

Description of component mark numbers

| | |
|---|---|
| 1 | Screw tail |
| 11 | Fixing column |
| 12 | Platform |
| 13 | Installation head |
| 14 | Installation adjusting hole |
| 2 | Screw seat |
| 21 | Rod groove |
| 22 | Locking thread |
| 23 | Screw seat sleeve |
| 24 | Slidable tensioning ring |
| 25 | Ball socket |
| 26 | Opening |
| 27 | Extension groove |
| 28 | Limiting ring |
| 29 | Loose section |
| 210 | Limiting groove |
| 211 | Limiting screw |
| 212 | Arc groove |
| 3 | Connecting rod |
| 4 | Locking bolt |
| 5 | Special screwdriver for |
| | holding screw tail |
| 6 | Vertebral body |

### DETAILED DESCRIPTIONS OF THE PREFERRED EMBODIMENTS

The implementation modes of the present invention will be described below through specific embodiments. One skilled in the art can easily understand other advantages and effects of the present invention according to contents disclosed by the description.

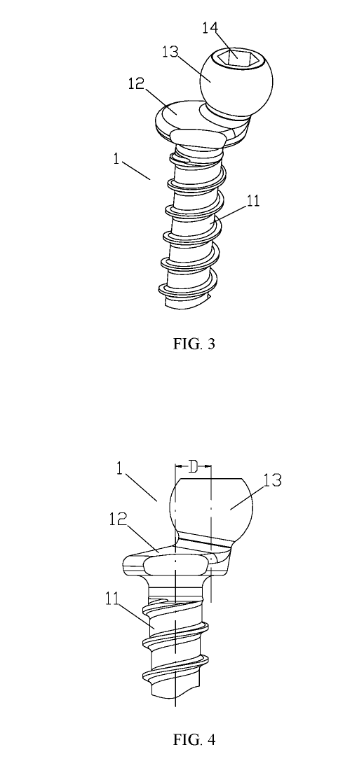

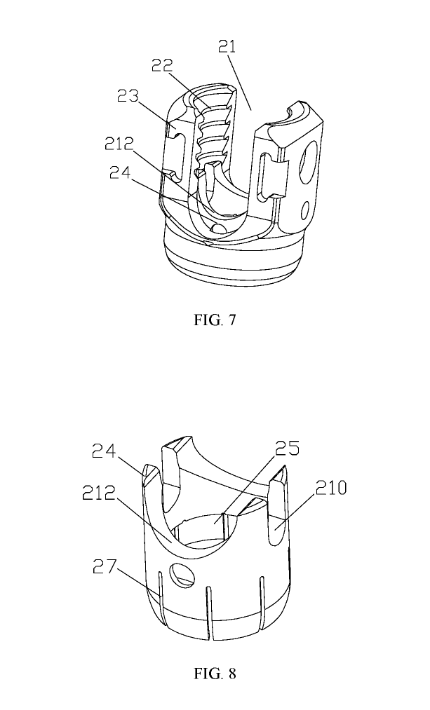

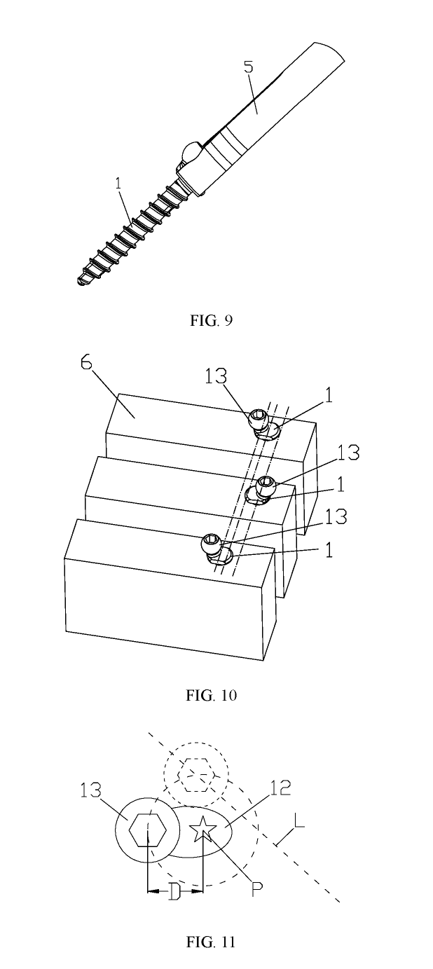

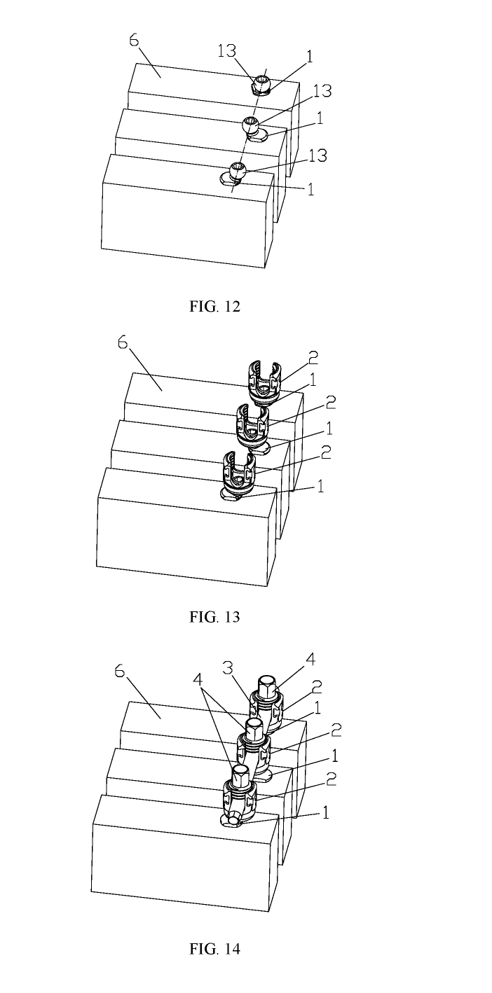

Please refer to FIG. 3 to FIG. 14. It shall be noted that the structures, scales, sizes and the like illustrated in the drawings of the description are only used for cooperating with the contents disclosed by the description to allow one skilled in the art to understand and read instead of limiting the implementable limitation conditions of the present invention, and thus have no technical substantive meanings; and any structural modifications, changes of scaling relations or adjustments to sizes shall still fall into the scope which can be covered by the technical contents disclosed by the present invention under the situation that the effects which can be produced by the present invention and the purposes which can be achieved by the present invention are not influenced. In addition, direction or quantification words such as “above”, “below”, “left”, “right”, “middle” and “one” cited in the description are just used for facilitating clear description instead of limiting the implementable scope of the present invention. Changes or adjustments of relative relations thereof shall also be deemed as the implementable scope of the present invention under the situation that the technical contents are not substantively changed.

As illustrated in FIG. 3 to FIG. 4, the present invention provides a transverse shift screw tail 1, the screw tail 1 comprises a fixing column 11 capable of being screwed into a vertebral body 6 and a platform 12 located at an end portion of the fixing column 11, the platform 12 is provided with an installation head 13 used for connecting with a screw seat 2, and an eccentric distance is provided between a center of the installation head 13 and an axis of the fixing column 11.

By adopting the eccentric installation head 13, the position of the installation head 13 can be adjusted by rotating the fixing column 11, such that the center of the installation head 13 is enabled to approach the reference line L (i.e., the placement position of the connecting rod 3) to the utmost extent, thus the screw seat 2 installed on the installation head 13 can be enabled to be located on the same line as much as possible, all screw seats 2 can be connected through one connecting rod 3, the spinal screws are located at the optimal positions on the spine and the spine is not caused to be improperly distorted.

A range of the eccentric distance D between the center of the installation head 13 and the axis of the fixing column 11 is 1 mm-5 mm, and the range of the eccentric distance D substantially satisfies a range that the screw tail 1 rotates for ¼ circle, such that the installation head 13 can approach the requirement of reference line L to the utmost extent.

In order to facilitate the fixation of the screw tail 1 into the vertebral body 6, an outer circumferential surface of the fixing column 11 is provided with a thread.

An outer surface of the installation head 13 is preferably a spherical surface and a top of the installation head 13 is provided with an inwards recessed noncircular installation adjusting hole 14. A cross section of the installation adjusting hole 14 is preferably hexagonal, and thus a hexagonal screwdriver can be inserted into the installation adjusting hole 14 and used for adjusting the position of the installation head 13.

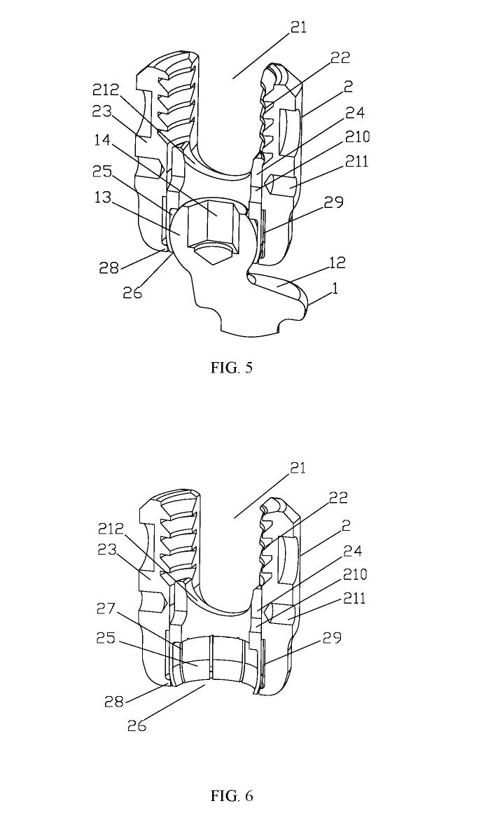

As illustrated in FIG. 5 to FIG. 8, the present invention provides a transverse adjustable spinal screw, comprising the above-mentioned screw tail 1 and a screw seat 2 installed on an installation head 13 of the screw tail 1, a top of the screw seat 2 is provided with a rod groove 21 used for placing a connecting rod 3, the rod groove 21 is recessed downwards from the top of the screw seat 2 and radially penetrates the screw seat 2, a bottom of the rod groove 21 is a curved surface adapted to an outer circumferential surface of the connecting rod 3, and a side surface of the rod groove 21 is provided with a locking thread 22 fit with a locking bolt 4.

Since the screw seat 2 has a certain transverse shift relative to the fixing column 11 of the screw tail 1, if the screw seat 2 is installed on the screw tail 1 in advance, when the screw tail 1 is rotated, the movement periphery of the screw seat 2 is larger and the operation is not facilitated. Therefore, the present invention provides a design of a screw seat, assembly that after the screw tail 1 is fixed on the vertebral body 6, the screw seat 2 is installed on the screw tail 1. The screw seat 2 installed on the installation head 13 is capable of rotating on the installation head 13 such that axis of the rod grooves 21 of a plurality of screw seats 2 form a line, and the connecting rod 3 can be placed in the rod grooves 21 to connect the plurality of rod grooves 21 together. In order to fix the connecting rod 3, a locking bolt 4 is installed at the top of the rod groove 21 and the locking bolt is fit with the locking thread 22 on the side surface of the rod groove 21.

One specific structure of the screw seat 2 is as follow: the screw seat 2 comprises a screw seat sleeve 23 and a slidable tensioning ring 24 which is sleeve-mounted into the screw seat sleeve 23, the slidable tensioning ring 24 is sleeve-mounted onto the installation head 13 of the screw tail 1, and the rod groove 21 is located at one end, far away from the slidable tensioning ring 24, of the screw seat sleeve 23.

The slidable tensioning ring 24 is provided with a ball socket 25 for placing the installation head 13, the ball socket 25 is provided with an opening 26 facing to the installation head 13, and a diameter of the opening 26 is smaller than a maximum diameter of the installation head 13. The installation head 13 can rotate at a plurality of angles in the ball socket 25 such that the screw seat 2 can be adjusted at a plurality of angles.

A circumferential surface of the ball socket 25 is provided with a plurality of extension grooves 27 which are uniformly distributed along a circumferential direction, and the extension grooves 27 extend from the opening 26 to one end corresponding to the opening 26. The ball socket 25 which is provided with the extension grooves is petal-shaped. In the process that the installation head 13 is placed into the ball socket 25 through the opening 26, by arranging the arrangement of the extension groove 27, enables the ball socket 25 to have certain elasticity and can be slightly outwards extended to guarantee smooth installation of the installation head 13; and when the connecting rod 3 is installed into the rod grooves 21, the connecting rod 3 will apply certain pressure to the slidable tensioning ring 24, the arrangement of the extension grooves 27 enables the ball socket 25 to have certain elasticity, and the length of the slidable tensioning ring 24 in an axial direction be properly changed to adapt to the installation of the connecting rod 3.

In order to prevent the slidable tensioning ring 24 from falling out of the screw seat sleeve 23, one end, facing to the screw tail 1, of an inner circumferential surface of the screw seat sleeve 23 is provided with a limiting ring 28 and an inner circumferential surface of the limiting ring 28 is fit with an outer circumferential surface of the opening 26 of the ball socket 25.

A portion, corresponding to the ball socket 25, of the inner circumferential surface of the screw seat sleeve 23 is provided with an inwards recessed loose section 29 and a gap is kept between the loose section 29 and an outer circumferential surface of the ball socket 25, so as to provide a space for deformation of the ball socket 25.

A side wall of the slidable tensioning ring 24 is further provided with at least one limiting groove 210 which extends downwards from a top, and the screw seat sleeve 23 is provided with a limiting screw 211 corresponding to the limiting groove 210. When the slidable tensioning ring 24 is inserted into a predetermined position in the screw seat sleeve 23, the limiting screw 211 is clamped at the bottom of the limiting groove 210 to lock relative positions of the slidable tensioning ring 24 and the screw seat sleeve 23.

One end, facing to the rod groove 21, of the slidable tensioning ring 24 is provided with an arc groove 212, an inner circumferential surface of the arc groove 212 corresponds to the bottom of the rod groove 21, and when the slidable tensioning ring 24 is installed in place, the inner circumferential surface of the arc groove 212 protrudes out of the bottom of the rod groove 21. After the connecting rod 3 is installed into the rod groove 21, the locking bolt 4 is installed from the top of the rod groove 21. Since the inner circumferential surface of the arc groove 212 protrudes out of the bottom of the rod groove 21, the connecting rod 3 will apply certain pressure to the arc groove 212 to enable the slidable tensioning ring 24 to move downwards, and at this moment, the ball socket 25 is deformed and then locks the installation head 13, so that the overall structure of the transverse adjustable spinal screw is more stable.

A method for implanting the transverse adjustable spinal screw into a vertebral body comprises the following steps:

1) determining a screwing-in point P of each screw on a vertebral body 6 and screwing screw tail 1 of each screw into the vertebral body 6 from the preset screwing-in point P;

2) setting a placement position of a connecting rod 3 on the vertebral body 6 and marking the placement position of the connecting rod 3 as a reference line L;

3) inserting a hexagonal screwdriver into an installation adjusting hole 14 and rotating the platform 12 to enable the center of an installation head 13 to approach the reference line L to the utmost extent;

4) putting the installation head 13 of each screw tail 1 into a ball socket 25 of a screw seat 2 to enable the screw tail 1 to be connected with a screw seat 2, rotating the screw seat 2 to enable axis of rod grooves 21 of all screw seats 2 to be located on the same line;

5) inserting the connecting rod 3 into the rod grooves 21 to enable all screw seats 2 to be integrally connected; and

6) installing the locking bolt 4 at the top of each screw seat 2, wherein the locking bolt 4 stretches into the rod groove 21 and is fit with the locking thread 22 in the rod groove 21, and at this moment, the connecting rod 3 will apply certain pressure to an arc groove 212, so that a slidable tensioning ring 24 moves downwards, and the ball socket 25 locks the installation head 13 after the ball socket 25 is deformed.

To sum up, according to the transverse shift screw tail, the transverse adjustable spinal screw and the implantation method provided by the present invention, by designing a brand new screw, the screw seats are connected linearly, the installation process of the connecting rod is enabled to be reliable and effective, the spine is prevented from being improperly distorted, the difficulty of operation by the doctor is reduced and the operation effect is improved. Therefore, the present invention effectively overcomes various disadvantages of the prior art and thus has a great industrial utilization value.

The above-mentioned embodiments are just used for exemplarily describing the principle and effect of the present invention instead of limiting the present invention. One skilled in the art may make modifications or changes to the above-mentioned embodiments without departing from the spirit and scope of the present invention. Therefore, all equivalent modifications or changes made by those who have common knowledge in the art without departing from the spirit and technical thought disclosed by the present invention shall be still covered by the claims of the present invention.

1. A transverse shift screw tail (1), characterized in that the screw tail (1) comprises a fixing column (11) capable of being screwed into a vertebral body (6) and a platform (12) located at an end portion of the fixing column (11), the platform (12) is provided with an installation head (13) used for connecting with a screw seat (2), an eccentric distance is provided between a center of the installation head (13) and an axis of the fixing column (11).

2. The transverse shift screw tail (1) according to claim 1, characterized in that an eccentric distance between the center of the installation head (13) and the axis of the fixing column (11) ranges 1 mm-5 mm.

3. The transverse shift screw tail (1) according to claim 1, characterized in that an outer circumferential surface of the fixing column (11) is provided with a thread.

4. The transverse shift screw tail (1) according to claim 1, characterized in that an outer surface of the installation head (13) is a spherical surface and a top of the installation head (13) is provided with an inwards recessed noncircular installation adjusting hole (14).

5. A transverse adjustable spinal screw, characterized in that the screw comprises the screw tail (1) according to claim 1 and a screw seat (2) installed on an installation head (13) of the screw tail (1), a top of the screw seat (2) is provided with a rod groove (21) used for placing a connecting rod (3), the rod groove (21) is recessed downwards from the top of the screw seat (2) and radially penetrates the screw seat (2), a bottom of the rod groove (21) is a curved surface adapted to an outer circumferential surface of the connecting rod (3), and a side surface of the rod groove (21) is provided with a locking thread (22) fit with a locking bolt (4).

6. The transverse adjustable spinal screw according to claim 5, characterized in that the screw seat (2) comprises a screw seat sleeve (23) and a slidable tensioning ring (24) which is sleeve-mounted into the screw seat sleeve (23), the slidable tensioning ring (24) is sleeve-mounted onto the installation head (13) of the screw tail (1), the rod groove (21) is located at one end, far away from the slidable tensioning ring (24), of the screw seat sleeve (23).

7. The transverse adjustable spinal screw according to claim 6, the slidable tensioning ring (24) is provided with a ball socket (25) for placing the installation head (13), the ball socket (25) is provided with an opening (26) facing to the installation head (13), and a diameter of the opening (26) is smaller than a maximum diameter of the installation head (13).

8. The transverse adjustable spinal screw according to claim 7, a circumferential surface of the ball socket (25) is provided with a plurality of extension grooves (27) which are uniformly distributed along a circumferential direction, and the extension grooves (27) extend from the opening (26) to an end corresponding to the opening (26).

9. The transverse adjustable spinal screw according to claim 7, characterized in that one end, facing to the screw tail (1), of an inner circumferential surface of the screw seat sleeve (23) is provided with a limiting ring (28) and an inner circumferential surface of the limiting ring (28) is fit with an outer circumferential surface of the opening (26) of the ball socket (25).

10. The transverse adjustable spinal screw according to claim 7, characterized in that a portion, corresponding to the ball socket (25), of the inner circumferential surface of the screw seat sleeve (23) is provided with an inwards recessed loose section (29) and a gap is kept between the loose section (29) and an outer circumferential surface of the ball socket (25).

11. The transverse adjustable spinal screw according to claim 6, characterized in that a sidewall of the slidable tensioning ring (24) is further provided with at least one limiting groove (210) which extends downwards from a top, and the screw seat sleeve (23) is provided with a limiting screw (211) corresponding to the limiting groove (210).

12. The transverse adjustable spinal screw according to claim 6 characterized in that one end, facing to the rod groove (21), of the slidable tensioning ring (24) is provided with an arc groove (212), an inner circumferential surface of the arc groove (212) corresponds to the bottom of the rod groove (21), and when the slidable tensioning ring (24) is installed in place, the inner circumferential surface of the arc groove (212) protrudes out of the bottom of the rod groove (21).

13. A method for implanting a transverse adjustable spinal screw into a vertebral body, the transverse adjustable spinal screw being the transverse adjustable spinal screw according to claim 5, characterized in that the method comprises the following steps:
1) determining a screwing-in point P of each screw on the vertebral body (6) and screwing the screw tail (1) of each screw into the vertebral body (6) from the preset screwing-in point P;
2) setting a placement position of the connecting rod (3) on the vertebral body (6) and marking the placement position of the connecting rod (3) as a reference line L;
3) rotating the platform (12) to enable the center of the installation head (13) to approach the reference line L to the utmost extent;
4) installing the screw seat (2) on the installation head (13) of each screw tail (1) and rotating the screw seats (2) to enable the axis of the rod grooves (21) of the screw seats (2) to be located on the same line;
5) inserting the connecting rod (3) into the rod grooves (21) to enable all screw seats (2) to be integrally connected; and
6) installing the locking bolt (4) at the top of each screw seat (2), the locking bolt (4) stretching into the rod groove (21) and being fit with the locking thread (22) in the rod groove (21).
